(19) United States
(12) Patent Application Publication
(10) Pub. No.: US 2003/0049626 A1
(43) Pub. Date: Mar. 13, 2003

(54) Antibody-based analysis of matrix protein arrays

(75) Inventor: Moncef Jendoubi, San Francisco, CA (US)

Correspondence Address: ORRICK, HERRINGTON & SUTCLIFFE, LLP, 4 PARK PLAZA, SUITE 1600, IRVINE, CA, 92614-2558, US

(73) Assignee: Milagen, Inc.

(21) Appl. No.: 09/930,715

(22) Filed: Aug. 14, 2001

(51) Int. Cl. C12Q 1/68; G01N 33/53; G01N 33/574

(52) U.S. Cl. 435/6; 435/7.1; 435/7.23

(57) ABSTRACT

The present invention is directed to a protein matrix wherein biological samples, preferably proteins, are placed on a solid support. The arrays are reacted with identifier molecules, such as antibodies, to correlate the gene expression manifested in the samples with the identifier molecule. The reaction produces a gene expression or protein expression profile for the biological state from which the samples are derived. The arrays are used for research, drug discovery, and the detection of disease.

### FIELD OF INVENTION

The present invention is a serial matrix of biological samples on a solid support where a molecular identifier, preferably an antibody, is used to bind target molecules to generate a profile of the samples. The invention is applicable to biological samples from pathogens, mammals, other animals and plants, and can be used for research, drug discovery and diagnostics.

### BACKGROUND OF THE INVENTION

Determining the sequence of the human genome provides information regarding the nucleotide content of the genome, but does not yield information about gene expression. Questions remain, however, as to how many transcripts and proteins are derived from each gene, what is the function of each protein, and ultimately, how each protein functions and interacts in the physiological context of normal and pathological conditions.

The life processes of a cell (i.e. growth, division, death, migration, shape, nutrition, metabolism, stress response, defense, etc.) are dynamic and reflect the cell's function as it constantly reacts to the environment. Each cell is capable of making a selection of specific proteins that vary in composition and in the level of expression depending on the cell and tissue type and on the environment in which the cell exists at a particular time. For example, the intrusion of a physical, chemical or pathological agent in the body immediately affects the subtle balance within the cell, and accordingly, may alter the type and quantity of proteins expressed. The response to an outside stimulus may turn on, off, or regulate gene expression, with changes in the amount of a transcript, gene product and modification of the protein. Also, in some circumstances, the agent or stimulus affects the cell structure or function to a degree sufficient to result in a disease state. In other cases, a disease may not be attributable to exposure to an agent or stimulus, but may be the result of other biological events whose cause or origin is unknown. However, whenever a disease is manifested in the body, the level of gene expression and protein production by certain cells is altered and this alteration can be measured to detect and diagnose the disease.

Scientists have devoted tremendous effort and resources to correlate disease processes with the underlying gene products.

Typically, when a gene product is suspected to play a physiological or a pathological role, researchers use an antibody, raised to react specifically with the gene product of interest, to further study and characterize the protein. For example, (1) the scientist identifies a gene product expressed differentially in a cell or a tissue type and which seems to play an important physiological role; (2) the scientist conducts biochemical studies to partially purify the suspected gene product, and immunizes an animal to make specific polyclonal antibodies to the gene product. Subsequently the polyclonal antibodies are used (a) to characterize the desired gene product; (b) to attempt to study the gene product function; and (c) ultimately, to screen an expression cDNA library in E. Coli to identify the gene sequence encoding the protein. There are numerous cases where the gene product and its sequence were discovered and validated based on this approach. In this example, the antibody was used as an identifier of the target molecule. Alternatively, similar molecules that specifically bind a target within a mixture of biological samples can also be used to identify the presence, the absence, or the amount of that target.

Because of the unique nature of the antigen-antibody reaction, antibodies have been used in a large number of different analytical or diagnostic methodologies to achieve a variety of analytical or diagnostic goals. The antibody provides the researcher with the ability to selectively bind the antigen and qualitatively or quantitatively detect the presence of the antigen in a sample and can be used to physically separate the antigen from a mixture of compounds contained in a sample. Thus, when a particular interest in a protein exists, the process of comparative analysis of that protein in several samples is an invaluable tool for studying the correlation of the protein with a physiological or a pathological onset.

Different strategies for the use of antibody-based immunoassays have led to a wide variety of applications wherein the antibody is used as a tool to characterize the protein target of interest. For example, an enzyme-linked immunoabsorbent assay (ELISA) is an assay technique that is particularly useful to diagnose antigens such as viruses, bacteria, or proteins, including other antibodies, in sera or in other biological samples. Particularly where quantitative analysis is desired, the antigen or antibody is absorbed onto a solid surface, such as the well of a microtiter plate, so that the reaction of a particular antibody-antigen pair can be analyzed. Using any of several variations on the ELISA, a quantitative or qualitative measurement of a particular antibody-antigen interaction can be made.

Thus, where the protein gene product is well characterized, antibody-based assays can be used to obtain information about that protein by relying on the specific reactivity with an antibody. However, where the protein is not well-characterized in structure or function, the situation is much different.

Recently, the prospect of sequencing the entire genome of an organism has led to a great hope for discovering new genes whose expression affects a variety of diseases or physiological conditions. However, given the large number of available genes, the absence of information regarding proteins encoded by these genes, and the absence of appropriate tools to characterize the gene products and their function, the analysis of gene function in actual physiological events remains a very slow process. The current strategies employed to interrogate biological processes and discover new genes related to pathological conditions fail to provide a rapid correlation between the gene expression product that may be obtained at the tissue or cellular level and the coding sequence. In addition, the current strategies remain costly and labor intensive. Thus, there is a great need to methods and processes to allow the accomplishment of a variety of comparative protein studies to correlate the physiological role of proteins to all manifestations of gene product expression.

Currently, the total number of genes in the human genome is estimated to be around 40,000 genes and possibly twice as much according to a recent publication (Fred A. Wright et al. Genome Biology, 2:7, 2001). The approximately 40,000 genes contained in the human genome are estimated to generate at least 5-10 fold more transcript isoforms and each transcript isoform is estimated to make several different proteins. Even when a gene is identified and correlated with disease, the correlation does not take into account the fact that multiple transcript isoforms are derived from each gene sequence or that many polypeptides may be made from each transcript. The absence of this information impedes the comparison, correlation and direct profiling of protein expression to disease states. Thus, genome sequencing information in and of itself does not yield important information about the expression of genes, including: (a) how many transcripts are made from each gene sequence, (b) how many isoforms are derived from each transcript, (c) how many open reading frame are contained in the same transcript, (d) which open reading frame is translated and which one is not, (e) how many polypeptides are derived from each transcript.

The prior art for protein analysis is based on the use of two dimensional acrylamide gel analyses, referred to as 2D-PAGE. This process allows the separation of proteins based on their size and electrical charges. This method has several drawbacks: (a) protein samples are usually denatured prior to their separation on 2D-PAGE; (b) the chemicals used in this process degrade certain proteins and modify others; (c) when different samples are subjected to 2D-PAGE, the individual samples are being modified differently by the process, which hinders an accurate comparative analysis of different samples; (d) the analysis of different samples requires different 2D-PAGE analyses and is labor intensive and time consuming. Because proteins are the ultimate pharmaceutical targets for diagnostic and therapeutics, there is a need to discover the gene products encoded by their corresponding gene sequences and to correlate their expression profiles in physiological and pathological states at the protein level. The need exists to correlate the gene product expression in different tissues or cell types, such as cancer cell lines, primary cell lines, and tissue specific cell lines at both the tissue and the temporal level. There is also a need to identify proteins whose expression is increased, decreased, initiated or terminated from an exposure of a certain cell type to any stimulus such as drug, hormone, chemical or physical agents.

There is a need to analyze thoroughly the expression products of the genome of an organism on the basis of their gene-products in different cell types, such as cancer cell lines, primary cell lines, and tissue specific cell lines. A differential screening of the gene products in these cell lines can be useful to identify specific gene-products expressed, up/or down regulated, or absent in these cell lines in normal and pathological conditions.

There is a great need to discover the gene products that are associated with a variety of threatening disorder such as cardiovascular disease, cancers, inflammatory, neurological and infectious diseases to discover the underlying gene functions and their relation with these diseases. Moreover, there is a need for a method that correlate the gene with its pattern of expression, in different cell types and tissues as well as their cellular localization.

Finally, there is a need to generate a data bank that contains all information related to mammalian gene products, their pattern of expression, gene-products and their isoforms, cellular expression at both the tissue and temporal level, and the gene sequences to which the gene products are correlated.

### SUMMARY OF THE INVENTION

The present invention takes advantage of: (a) the specific interaction between two molecules, preferably antibodies, and a target protein in a mixture of a biological samples; (b) the efficiency, the specificity, and the wide immune response against a particular polypeptide of any length and form, including peptides, proteins, protein fragments, carbohydrates, organic molecules, or any immunogen against which the immune system of an animal will produce antibodies; (c) a process to create a matrix protein array from different biological samples of various physiological conditions and the possibility to analyze them under the same condition simultaneously; (d) the need to analyze gene expression and to discover new genes and new gene expression products, particularly proteins having physiological significance in human disease; (e) the exquisite nature of specificity of the antibody and their broad use in multiple and diversified assays, including multiplex format for rapid high throughput analysis of biological samples; (f) the possibility to correlate the antigen present in a biological sample with the coding gene sequence.

The present invention uses antibodies to known and unknown polypeptides to analyze many biological samples contained in the same physical area, using one antibody for each physical area. This process will allow a large scale differential, comparative analysis of protein expression for a number of biological samples at the same time and leads to high throughput capabilities for discovery and validation of pharmaceutical disease targets. One advantage of the present invention is that the antibody will interact with all samples under exactly the same conditions and the antibody-target binding reaction yields detailed information about gene expression in each sample. The present invention includes the immobilization of gene products on a solid support, preferably in an array or matrix for high throughput analyses, gene product discovery, and validation. The present invention also includes the use of a gene product array for high throughput testing by antibodies obtained pursuant to the practice of this invention.

The invention also enables the use of antibodies to conduct gene product expression profiling of a given normal or diseased tissue, or biological samples from a patient with disease or suspected of disease, for analyzing a cell type before and after exposure to drugs, chemicals, or physical stimuli, such as carcinogens, irradiation, toxic agents, pharmacological agents, and the like. Antibodies may also be used to discover gene products related to any chemical, toxic, or physical agent and/or any other stimulus, to an onset of disease, the progression of disease, the resistance to treatment, or the relationship between toxic agents and abnormally regulated gene products, or virtually any pathological and physiological changes characteristic of a normal or disease state.

Thus, the matrix protein arrays of this invention allow the examination and analysis of hundreds of gene products simultaneously, and reflect a specific physiological or pathological state of the sample, whether derived from cells, tissues or biological fluids from a host. The host, for example a human patient, may be in a normal or disease state, or exposed to a stimulus or chemical or biological agent. In any case, this high throughput analysis provides important advantages over methodologies that examine only one or few gene products at any given time.

Furthermore, the present invention also includes matrix protein array screening in multifunctional assays. The present invention also identifies and correlates in one-to-one relationships, the antibody, the gene product and the physiological or pathological outcome.

### DESCRIPTION OF THE FIGURES

FIG. 1 is a schematic of a matrix protein array used to react 96 antibodies with samples in each compartment of columns I-XII and rows A-H. Compartment A XII contains 16 biological samples of two types for differential analysis with antibody no. 12.

FIG. 2 is a schematic of a matrix protein array used to react 96 identifier molecules in each compartment of columns I-XII and rows A-H. Compartment A XII contains 16 biological samples of 4 types for differential analysis with identifier molecule no. 12.

FIG. 3 is an embodiment of the matrix protein array with a plurality of biological samples oriented in each compartment.

Note: The patterns and markings in the Figures are used to represent different biological samples.

### DETAILED DESCRIPTION OF THE INVENTION

“Peptide,” “polypeptide,” “gene product,” or “gene expression product” comprise the broad class of compounds that are produced by transcription and translation of a polynucleotide sequence, regardless of its length, in a cell. These translation products include peptides, proteins, polypeptides, and may include post-translational modification, such as conjugation with a lipid, a phosphate, a sugar, or the like. The polypeptide may be a full-length protein exhibiting normal folding patterns, as well as fragments and other forms of polypeptides produced by post-translational events.

The term “antibody” refers to a binding protein having reactivity with an antigen and includes monoclonal, polyclonal antibodies, and antibody fragments having specific binding affinity to a peptide, a polypeptide, gene product or a fragment of the gene product of a cell.

The term “antibody fragment” refers to a portion of an antibody, often the hypervariable region and portions of the surrounding heavy and light chains, which display specific binding affinity for a particular molecule. A hypervariable region is a portion of an antibody that physically binds to the polypeptide target. The antibody molecule is a glycoprotein comprising at least two light polypeptide chains and two heavy polypeptide chains, wherein each light and heavy chain contains a variable region located at the amino terminal portion of a polypeptide chain featuring an antigen-interaction region wherein the antigen is bound. The heavy and light polypeptide chains are also comprised of a constant region at the carboxy terminal portion.

“Monoclonal antibodies” are substantially homogenous populations of antibodies to a particular antigen. They may be obtained by any technique that leads to the production of antibody molecules by continuous cell lines in culture. Monoclonal antibodies may be obtained by methods known to those skilled in the art. See, for example, Kohler, et al., Nature 256:495-497 (1975), and U.S. Pat. No. 4,376,110.

The term “polyclonal” refers to antibodies that are heterogeneous populations of antibody molecules derived from the sera of animals immunized with an antigen or an antigenic functional derivative thereof. For the production of polyclonal antibodies, various host animals may be immunized by injection with antigen. Various adjuvants may be used to increase the immunological response, depending on the host species.

By “an array of antibodies” or “antibody array,” it is meant a group of antibodies bound to a solid support, where at least two of the antibodies in the array are directed to different binding partners. The antibodies are preferably arranged to form a line. However, the antibodies may also be arranged in any other formation, such as in a circle, a semi-circle, or to form shapes, such as X, [symbol] , or +, or any other shape.

By “specific binding affinity” is meant that the antibody binds to target polypeptides with greater affinity than it binds to other polypeptides under specified conditions. Antibodies having specific binding affinity to a gene product may be used in methods for detecting the presence and/or amount of the gene product in a sample by contacting the sample with the antibody under conditions such that an immunocomplex forms and detecting the presence and/or amount of the antibody conjugated to the gene product.

“Identifier” refers to a molecule that binds specifically to a target in a sample. Preferably the identifier is an antibody or peptide, a mimotope, an organic molecule, or other biological tag having a defined reactivity with another molecule to form a binding pair whose reaction identifies a molecule.

“Matrix protein array” refers to a total protein extract from biological samples from the same type, different types, the same species, different species, the same physiological conditions, different physiological conditions or any combination of the above arrayed on a solid support contained in a defined physical space which is separated from others. The number of each type of total protein extract may vary from 2-10,000 different types of extracts forming the matrix.

The cells, tissue samples, biological fluids or derivatives or extracts of these that are used in the invention are from an organism, either single cell organisms, such as bacteria, viruses, amoebae, or protozoa, or multicellular organisms, such as members of the plant and animal kingdoms. The organism is preferably a plant, an insect, or an animal. The plant is preferably selected from the group consisting of crops, such as grains, nuts, vegetables, and fruits, household plants, trees, and bushes. In most diagnostic applications, the sample is from a human patient and may include tissue from a normal or disease state such as a tumor, a biological fluid such as ascites, urine, plasma, serum, spinal or cerebral fluid, or other preparation and may be processed for advantageous use in the kits of the invention.

The present invention includes:

a) the creation of a matrix of protein arrays from different biological samples contained in a physical space, and where the biological samples can be from the same type of tissues, different tissues, normal and disease tissues, different cell types, stimulated cell types with chemical and/or physical agents, or a mixture of any of the above.

b) the matrix of the protein arrays, regardless of the origin of the biological sample, can be repeated several times with the same content of the different matrix protein or with different content of matrix proteins.

c) applying antibodies, their derivatives or identifier molecules in multiplex formats on matrix protein arrays of biological samples, so that the gene products bind to their respective antibodies in the matrix protein arrays, and where the binding can be measured to analyze biological samples to gain valuable information from the binding event;

d) selecting antibodies against known and unknown gene products that correlate to a disease onset, a biological process or any other relevant biological information;

e) determining the presence or absence, and if present, the amount of, the gene products bound to said antibodies; and

f) correlating the results to the presence or absence of disease or to a relevant biological function.

In preferred embodiments, the biological sample is derived from a human patient who has or is suspected of having a certain condition. The certain condition may be any disease or physiological state, including a state of progress of a specific disease, disease susceptibility or resistance, or any response or resistance to treatment. The gene products may be obtained from any source of tissue or sample as described herein.

In another specific aspect of the invention, a disease is diagnosed in an organism by: (a) creating the matrix of protein arrays on a solid support; (b) using antibodies, their derivatives or other identifier molecules to profile biological samples, by contacting cell lysates from the organism to be analyzed using array of antibodies against matrix of protein arrays of the targeted mammal or pathogen, so that the gene products bind to their respective antibodies in the array, where the binding can be measured and compared to a standard or to values indicating a normal or disease state.

Preferably, the biological assay intended for the diagnostic could be performed to diagnose human diseases or infectious pathogens (e. i. amoebae, fungi, viruses, and bacteria and the like).

### EXAMPLE 1

### Concept and Description of the Matrix Protein Arrays

The solid support used to create the matrix protein arrays is composed of different physical areas and can be referred to as wells, compartments, units, surfaces, physical areas and the like. The solid support can be made of plastic, glass, nitrocellulose, ceramic or any other suitable material contemplated for this purpose.

The solid support of the matrix protein arrays is distributed on two axes “X” and “Y,” or “X”, “Y” and “Z” representing a surface unit or a compartment respectively. Each physical area of the matrix protein arrays is referred to by a number. Physical areas are separated from each other. Each physical area may contain several biological samples from various origin or physiological conditions.

Solid support: the solid support contemplated in this invention to contain the biological sample to create the matrix protein arrays could be of any of the following material cellulose, nitrocellulose, nylon, ceramic, glass, polystyrene, etc., without limitation to the use of other solid supports. Alternatively the solid support of this invention refers to a device composed of two parts, a base and a divider. The assembly of these two parts allows the creation of compartments or wells of different size and total numbers, as the divider permits. The number of compartments created by this process may vary from 2-1,000 or from 2-10,000 or any number in between or more than 10,000 compartments if needed. This process creates individual compartments tightly separated from each other, such that when each compartment is loaded with different solutions, the different solutions will not mix with each other. The biological sample is either spotted directly on the lower base of the device or on a solid support of choice which can be tightly adjusted between the lower and the upper divider part of the device. Alternatively, the solid support could be glass or plastic divided by a thin layer of plastic, glue or other substance that would allow the creation of separate compartments.

In a preferred embodiment of this invention, the total protein extracts are immobilized in a rationally organized matrix on a solid support. Examples of such solid supports include, but are not limited to, plastics such as polycarbonate, complex carbohydrates such as agarose and sepharose, acrylic resins and such as polyacrylamide and latex beads. The solid support may also include glass, silica, silica gel, silicon wafer, silicone, plastics such as polyethylene, polystyrene, polyvinyl chloride (PVC), or polyvinyl pyrrolidone (PVP), nylon, TEFLON®, nitrocellulose, ceramic, fiber optic, and semiconductor materials. Techniques for coupling antibodies to such solid supports are well known in the art (Weir et al., “Handbook of Experimental Immunology” 4th Ed., Blackwell Scientific Publications, Oxford, England, Chapter 10, 1986; Jacoby et al., Meth. Enzym. 34, Academic Press, N.Y., 1974).

The matrix protein arrays comprise a group of individual total protein extracts, which group comprises at least two distinct samples of total protein extracts and preferably comprises a much larger number including values between 10 and 100 and integral values therein, values between 100 and 1,000, in integral values therein, as well as intervals of 10, values between 1,000 and 10,000, as well as integral values therein, and intervals of 10 or 100, and values between 10,000 to 100,000, and as high as a protein to each expression product in vertebrate, plant, or insect genomes, as well as integral values therein in intervals of 10, 100, 1,000, and 10,000. In preferred embodiments of the invention, therefore, at least two of the extracts are different and in preferred embodiments at least 10, or at least 100, or at least 1,000, or at least 10,000, or at least 100,000 of the extracts have different specficities, and are reactive with different gene products. As above, all integral values and intervals between these ranges are expressly disclosed herein. Furthermore, any numerical value in the above ranges corresponds to the number of extracts that are bound to a solid support to form the particular arrangement of the array.

The extracts may be arranged in a number of different ways on the solid support. For example, they may be arranged to form a line, a semicircle, a circle, a X, a [symbol] , or a +, or any other shape or combination of shapes. Preferably, duplicate quantities of the protein are arranged in a side-by-side fashion in the array to provide for reproducibility and a control. For purposes of diagnostics, the arrays may be organized on a physiological basis such that individual portions of the arrays reflect differential expression in different disease states. For example, an array may be constructed having gene expression products for a variety of diseases, wherein such expression products are known to be expressed in a biological fluid such as saliva, urine, serum, plasma, ascites, spinal or cerebral spinal fluid, etc. In such a rational organization, the location of binding events of antibody target (s) in a sample provides specific information about the physiological state of the underlying organism.

Using the matrix embodiment of this invention, an array of biological samples are located on a solid support within each compartment created by a device. The biological samples of the matrix can vary in numberfrom 2-1,000 or from 2-10,000 samples or more if needed. Biological samples can be from the same type and physiological condition, or they may differ in number, type and condition, as well as being derived from the same species or from different species. As it is understood by those skilled in the art, any combination or permutation of the biological samples is contemplated herein whether specifically disclosed in the preferred embodiment or not. Thus, the total number of matrix protein arrays within any device is referred to herein by matrix protein arrays and can be composed of 2 compartments, 10,000 compartments or any number of compartments.

Identifiers are reacted with the matrix arrays. The identifier molecule is preferably an antibody, whether polyclonal, monoclonal, combinatorial, single chain, or Fab antibody. Alternatively, the identifier is a chimeric peptide, or a combinatorial chemical molecule. Typically, a solution containing a distinct identifier is added to each compartment containing the protein arrays creating a multiplex combination in each compartment based on the following factors: a matrix of biological samples, number of biological samples, source of biological sample identified by various conditions from which it is derived, and different identifiers, principally different antibodies having different specificities. Importantly, the antibodies used in the arrays are correlated to gene products such that when the antibody functions as an “identifier,” the reaction indicates gene expression in the biological sample. Subsequently, the reaction complex of the identifier and its specific target is identified using a detection substance. Any variation, permutation and combination of these factors are contemplated in the practice of this invention and may be included accordingly whether or not specifically disclosed herein.

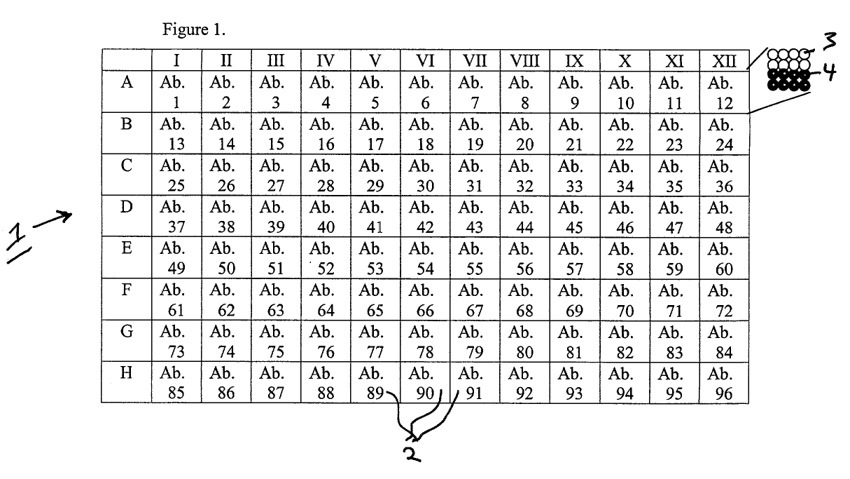

For example, as represented in FIG. 1, the solid support 1 containing the matrix protein arrays may be composed of 96 separate compartments 2. In this embodiment, each compartment contains the same type, number and composition of biological materials. In this example, 8 samples are derived from normal cells 3 (represented by white circle in the drawing) and 8 are derived from diseased 4 samples (represented by dark circles in the drawing). Each biological sample from disease and normal may correspond to total protein extract, processed cell derivatives, tissues or any sample to be interrogated in high throughput fashion. Different identifiers, preferentially antibodies, are used to analyze the matrix protein arrays and to secure data for the differential reactivity of each sample with each antibody.

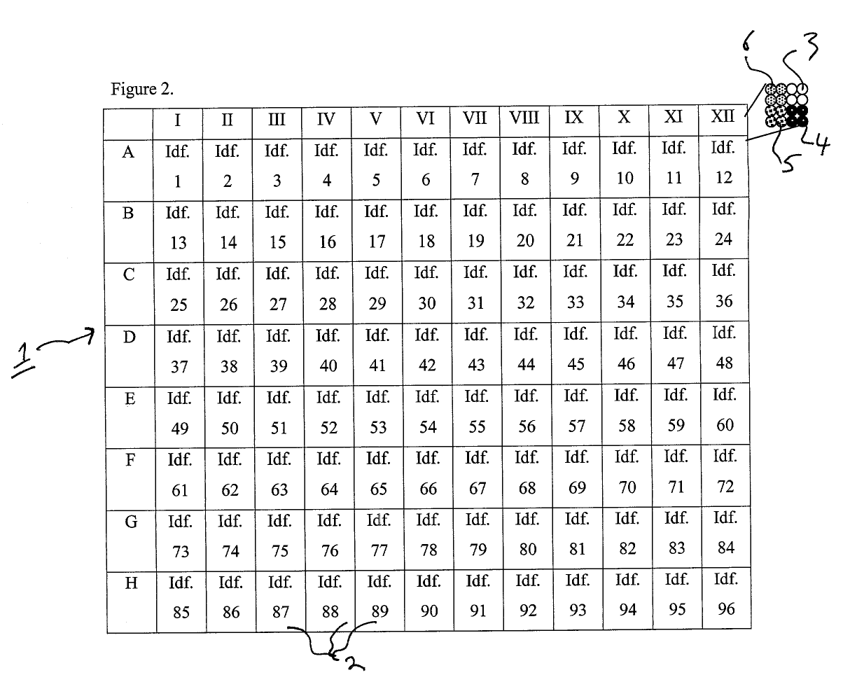

Referring to FIG. 2, as with the antibodies in the embodiment of FIG. 1, the discrete identifier molecules are exposed to each compartment containing biological samples for differential analysis. As noted above, while the number of compartments 2 comprising the matrix protein arrays may vary, a single compartment 2 can be comprised of any combination of biological samples 3, 4, 5, 6. For example, the protein in the biological samples 3, 4 are derived from at least two, or more, optimally distinct physiological states. For example, the compartment may contain samples derived from a normal 3 patient, an early stage cancer 4, a more progressed vascularized tumor 5, and a diffuse metastasized malignancy 6. By reacting a large number of samples with discrete identifier molecules a protein expression profile is created based on the identity of the reaction between the sample and the identifier molecule. Also, the distinct character of the samples may be established in many ways, for example, the sample may contain the same type of the sample tissue from different individuals, the same type of tissue at different developmental stages of the same individual, the same type of tissue in different pathological or physiological conditions, the same cell type exposed to a biological, chemical or physical stimuli during period of time. The matrix may contain the same type of tissue from different species. The matrix protein arrays preferably receive distinct identifiers in each compartment leading to direct information of protein expression profiling for each member of the matrix.

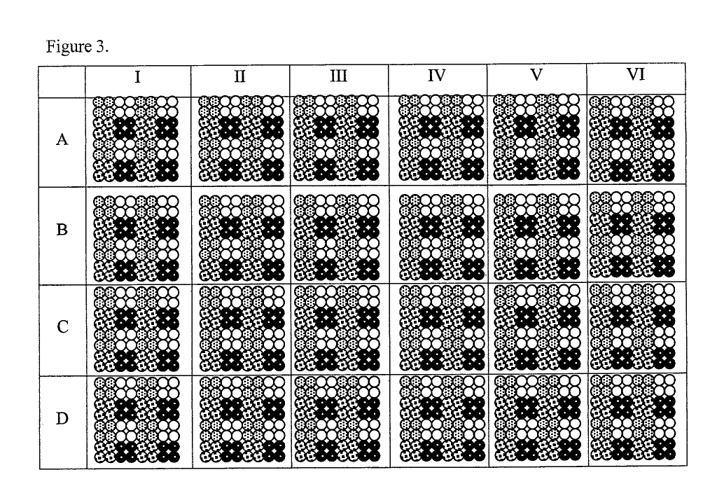

When the matrix protein array is used for screening of different biological samples or for their physiological and pathological conditions, the compartment of the solid support for the matrix protein arrays may vary in size and shape to accommodate the placement of a large number of samples in one compartment as illustrated in FIG. 3.

It is understood by those skilled in the art that the present invention allows all kind of combination of biological samples, number of samples, conditions of the samples, size of the compartment of the matrix protein arrays, type of identifiers, or any permutation of the above.

### EXAMPLE 2

### Preparation of the Matrix Protein Arrays and its Analysis with Identifiers

Extraction of protein from tissues: Fresh or frozen tissue from, for example humans, is cut off in small pieces, grounded, homogenized in an appropriate solution (Tris-HCl, pH7.5, 50 mM, EDTA 2 mM, NaCl 100 mM, NP40 1%, and vanadate 1 mM, in addition to the following proteases inhibitors PMSF, Aprotinin, Leupeptin at 1, 2 and 4 mM respectively), kept on ice for 20 minutes and centrifuged at 14,000 rpm for 15 minutes. The supernatant is transferred to a new container and the pellet of the tissue is resuspended, kept on ice an additional 20 minutes and centrifuged as indicated above. The supernatants are combined and the protein concentration is determined according to standard conditions as known to those skilled in the art. Protein solutions are kept in a −80 degree C freezer until further usage.

Extraction of protein from cells: Mammalian cells (about 10×10⁶) are grown in culture in DMEM, fetal calf serum 10%, Streptomycin and penicillin 100 μg/ml until 80% confluency, harvested, washed twice with PBS, resuspended in phosphate buffer (pH 8.0) and disrupted in the following buffer: 50 mM, Tris-HCl, pH7.5, EDTA 2 mM, NaCl 100 mM, NP40 1%, and vanadate 1 mM, in addition to the following proteases inhibitors PMSF, Aprotinin, Leupeptin at 1, 2 and 4 mM respectively. The cell lysate is centrifuged for 5 minutes at 14,000 rpm, and protein content is determined by standard method.

Printing of total protein extract: Two layers of precut blotting paper are placed in and Omni-tray and soaked with TBS solution. A precut sheet of nitrocellulose is placed over the blotting paper. The individual samples of the matrix protein arrays may be deposited manually or with a Robotic system (Genomic Solutions Flexys™) to construct the array. The matrix protein arrays are dried on blotting papers for 5 minutes at room temperature. The solid supports containing the matrix protein arrays are rinsed twice in TBS solution with rocking (2 rinses of 5 minutes each), treated with blocking buffer in blocking solution (2.5% Non Fat Dry Milk in TBST) in a wide tray for 1 hour with constant rocking at room temperature. After blocking, the matrix protein arrays are given 2 quick rinses with TBS solution for 2 minutes each. Total protein extracts can be printed either manually or using any robotic system as known to those in the art.

Antibodies: antibodies are routinely used at 1:1,000 dilution or more when needed. Dilution buffer is composed of Tris buffered saline (TBS) containing 0.02% BSA, 0.02% sodium azide. Aliquots of each antibody solution are added to each compartment containing a matrix of protein arrays.

Detection of the complex (antibody-gene product target): The solid support containing the matrix protein arrays is washed five times in the same buffer described above and incubated with sheep anti-mouse IgG antibodies conjugated with horseradish peroxydase, diluted 1/1000 in the blocking solution for 1 hour. Then, matrix protein arrays are washed 5 times 10 minutes each in TNE/Tween without milk. Anti-antibodies were visualized using the alkaline phosphatase conjugated substrate solution (Pierce named SuperSignal™ CL-HRP Substrate System). Matrix protein arrays are exposed to X-OMAT AR film for several seconds to several minutes to reach a satisfactory detection of the specifically recognized gene products.

A chemiluminescent substrate is prepared by mixing equal volumes of the two solutions in a 50 ml polypropylene tube. About 7 ml of substrate solution are prepared for each immunoblot. The solutions are mixed in a dark room to avoid light. Seven ml of substrate solution are dispensed in an Onmi-tray and the immunoblot is placed in the tray, followed by incubation in the dark by rocking for 5 minutes on a Vertical rocker. After 5 minutes of incubation, the blots are placed in a plastic sheet cover and the sheet is placed in an x-ray cartridge. The film is replaced for various progressive times to gauge the appropriate exposure time (from few minutes to 30 minutes) to achieve dark spots without complete saturation of the spots.

Alternatively, other protocols using different labeling and detection systems can also be used within the scope of the present invention.

### EXAMPLE 3

### Time Course of Gene Product Expression Profiling Following the Action of a Stimulus

In a preferred embodiment, the present invention is used to monitor changes endured by an organism that occur over time, or which result from an exposure to a stimulus such as biological agent, physical agent or chemical agent or a mixture of the above. For example, once organisms are exposed to a stimulus, the stimulus affects the subtle balance of the expression of a variety of gene products of the organisms. For example, the humans exposed to extensive UV irradiation gradually manifest detectible changes in physiology, which in some cases result in melanoma cancer. In other cases, when individuals are suffering from cancer and are being subjected to chemotherapy regiments, they often suffer from side effects. In other cases the chemotherapy become less effective over time rendering the treatment of the patient more difficult. In these examples (onset of melanoma cancer, side effect and inefficient treatment) the body undergoes a number of physiological changes at the protein level, all of which result in an undesirable effect. The protein matrix array pinpoints the gene products whose changes are coincident with the undesirable effect. The information derived from the arrays identifies the molecules involved in the underlying physiological changes that occur with a given stimulus.

In a preferred embodiment, the present invention monitors molecular changes over time following the exposure to a given stimulus. To achieve this process, a biological sample is taken at different time intervals from a cell, or a tissue of the organism, following the exposure to a given stimulus. The total protein extract from the different biological material removed at different times is used to create matrix protein arrays. Each matrix would contain the total protein extract from the different time intervals in each compartment. A different identifier is then added to each compartment. The identifier binds to its target whenever it is encountered in each total sample in the matrix resulting into a database of gene product expression profiling over the time course of the stimulus. This process will establish a correlation between the stimulus, the varying gene products, and the resulting physiological or pathological conditions.

Alternatively, the stimulus could be a hormone, a growth factor, a biological agent, a chemical or a physical agent or any combination of the above.

### EXAMPLE 4

### Target Discovery of Gene Product in Normal Diseased Biological Samples

In a preferred embodiment, the present invention can be applied in parallel to biological samples from diseased and normal tissues. In this case, a large number of different identifiers, preferably antibodies, are applied to the same type of biological samples of the at least two conditions (for example 10 normal versus 10 diseased samples) on the same matrix protein array. Identifiers will bind specifically their targets in the biological samples in each array leading to distinctive recognition of disease targets. For further validation, specific disease target identifiers are selected and reanalyzed on a larger number of the same type of biological samples.

It is known in the art that in response to a condition or a disease, differential expression at specific genes of an organism occurs, giving rise to the presence of specific gene products in the organism's cells. For example, if an organism suffers from a viral or a bacterial infection, to combat or cope with the infection, the organism produces certain gene products. It is also known that the organism may produce the gene products specific to the condition before the organism itself shows any morphological signs of suffering. By way of example only, a person suffering from the common cold will produce specific gene products associated with the disease before the person notices a runny nose or watery eyes.

Similarly, in carcinomas, the up or down regulation of genes that cause or accompany the disease state will result in the differential expression of genes and the differential presence of gene products in a sample. The gene products contained in virtually any biological sample may be described as “cell contents” because such products are excreted, derived, or extracted from a cell source such as tissue, plasma, tumor cells or tissue, etc. To test whether an organism is suffering from that disease or condition, the cell contents are exposed in matrix protein arrays to identifiers, preferably antibodies, to examine and correlate the disease event with specific gene targets. The binding events at the reaction sites of the antibody to its target in the matrix enables the identification of the gene products excreted, derived, or extracted from the cell. Matrix protein arrays can be used to differentially examine cell types, tissue types, and disease types, for toxicology, pharmacology, chemopharmacology, exposure to any physical or chemical agent, etc.

In each case, practice of the invention enables one to identify the differential expression of target proteins that comprise both the causative agents of the underlying disease state, as well as physiological events that are components of the overall biological cascade resulting from any disease state or exposure to stimulus. Once the differential expression profiles are obtained, the gene sequences and corresponding gene products can be identified and further studied for use as markers, as well as diagnostic or therapeutic products.

### EXAMPLE 5

### Validation of a Disease Target in an Animal Model Using a Matrix Protein Array

A variety of gene products are conserved among different animals and play similar physiological roles in each species. Over the years, scientists used these animals as models for human diseases to accelerate the discovery and correlation of gene products to the disease process. In an embodiment of the present invention, antibodies to human proteins are applied to matrix protein arrays containing proteins from different tissues and disease states to yield information on the gene expression profile of human disease.

In a preferred embodiment of the present invention, the disease target is comprised of protein found in human tissue types as referred to in example 3. The identifiers, preferably antibodies, that specifically recognize the disease targets are used to analyze matrix protein arrays containing biological samples from an animal model manifesting the same disease type. In this case, the total protein extract from the same type of tissue as that of the human are prepared from normal and diseased tissues of an animal. The animal samples are used to create an animal model matrix protein array to which selected antibodies recognizing disease targets in humans are applied. The information obtained from such an array is the same as is described elsewhere herein, however, the gene expression profile measures the animal model and can be analyzed as representative of the human disease.

The antibodies of the present invention include monoclonal and polyclonal antibodies, as well as fragments of these antibodies, and humanized forms. Sera containing the antibodies is screened for the presence of antibodies with the specific binding of a given gene product. Specific antibodies to a given gene product in the assay can be detected using an anti animal antibodies. Procedures for accomplishing such detection labeling are well known in the art.

As noted above, an important feature of the present invention is that the antibodies or identifiers, when tested on a biological sample, will react against the specific target, if it is present in the biological sample within the matrix protein arrays. Thus, using the present invention, antibodies may identify known or unknown proteins encoded by polynucleotide sequence to known or unknown genes. By observing the reaction between the antibodies and their targets in a sample, the antibodies provided by this invention can be used to explore the function of known or unknown proteins and related compounds. For example, the expression of a gene encoding an unknown protein may be studied in normal versus cancer clinical samples. When larger number of gene product identifiers are used, the identifiers can be rationally organized and tested against samples from known sources to characterize differential expression of a multitude of individual proteins in, for example, normal versus diseased samples. The reaction of the proteins with the individual members of a rationally organized identifier array also leads to identification of a large number of gene product that are differentially expressed in the normal versus diseased state. Similarly, differential comparative analysis gene expression in various cell types, tissue types, and developmental stages can be obtained pursuant the application of the matrix protein arrays to two physiological states differentiated by virtually any state or stimulus. When a polyclonal sera with a particular reactivity of interest, such as reactivity to a cancer-specific protein is identified, monoclonal antibodies may be produced that display the reactivity of interest. Labeling and detection system of antibodies reactivity are well known to those in the art of this field.

### EXAMPLE 6

### Identification of Antibody Targets of Specific Pathways

To analyze pathway differential gene expression in response to external stimulus, normal and diseased biological samples are analyzed by exposing the proteins of the sample to arrays of antibodies. The selected antibodies used to construct the array may or may not be specially selected for the indication. For example, using the same protocol, the gene expression of varying cell types can be analyzed following exposure to hormones, growth factors, bioactive chemicals generally, drugs, especially chemotherapy compounds, and virtually any toxin or agent whose effect on cell growth or metabolism and the underlying gene expression is of interest.

### EXAMPLE 7

### Identification of Antibody Targets

Antibodies have been used for many years for protein capture, validation, identification, separation from a biological mixture, etc. An important embodiment of the present invention is the use of the antibodies to further identify their bound targets without due need for characterization of the antigen proteins.

For example, the antibodies recognizing a target of interest can be used to capture its target from a protein mixture of a biological sample using immuno-affinity techniques known by those skilled in this art. The complex of antibodies-protein target of interest can either be used to immunize a mammal to make a large supply of antibodies, including the use of a mouse to generate monoclonal antibodies as described above. In another embodiment of the present invention, captured targets are analyzed by mass spectrometry to determine the primary amino acid sequence.

Antibodies can also be used to screen an expression library of genomic or cDNA from particular tissues or cell types. Specifically identified clones through this experiment are selected and the polynucleotide sequence is determined as known to those skilled in the art.

All patents and publications mentioned in the specification are indicative of the levels of those skilled in the art to which the invention pertains. All patents and publications are herein incorporated by reference to the same extent as if each individual publication was specifically and individually indicated to be incorporated by reference. It should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method to analyze gene expression comprised of:
providing a plurality of samples of biological material arranged in a discrete compartment, wherein the plurality of samples contain gene expression products derived from at least two distinct biological conditions,
contacting each of the plurality of samples with an antibody wherein the antibody is specific to the expression product of a gene sequence, and
correlating the reaction between the antibody and the plurality of samples with expression of the gene sequence.

2. The method of claim 1 wherein the antibody is a murine polyclonal antibody.

3. The method of claim 1 wherein the step of contacting the plurality of samples is performed with at least 100 antibodies.

4. The method of claim 1 wherein the plurality of samples of biological material is comprised of samples from a human disease.

5. The method of claim 1 wherein the sample of biological material is a protein extract.

6. The method of claim 5 wherein the protein extract is derived from cancer cells or tissue.

7. The method of claim 1 further comprising the step of repeating the contacting step to identify a plurality of gene sequences associated with a biological condition.

8. The method of claim 7 wherein the biological condition is cancer.

9. The method of claim 7 wherein the biological condition is exposure to a chemical agent.

10. The method of claim 7 wherein the step of identifying a plurality of gene sequences is a differential analysis between a normal and disease state.

11. The method of claim 1 further comprising identifying the expression product of the gene sequence.

12. The method of claim 11 further comprising raising a monoclonal antibody to the expression product of the gene sequence.

13. The method of claim 1 further comprising determining the polynucleotide sequence of the gene sequence.
